(19) United States
(12) Patent Application Publication
(10) Pub. No.: US 2005/0038388 A1
(43) Pub. Date: Feb. 17, 2005

(54) Injection device comprising an energy storage device

(76) Inventors: Edgar Hommann, Grossaffoltern (CH); Beat Steffen, Saanen (CH)

Correspondence Address: David E. Bruhn, Esq.;DORSEY & WHITNEY LLP, Intellectual Property Department, 50 South Sixth Street, Suite 1500, Minneapolis, MN, 55402-1498, US

(21) Appl. No.: 10/825,865

(22) Filed: Apr. 16, 2004

Related U.S. Application Data
(63) Continuation of application No. PCT/CH02/00563, filed on Oct. 15, 2002.

(30) Foreign Application Priority Data
Oct. 18, 2001 (DE) DE 101 51 471.9

(51) Int. Cl. A61M 37/00

(52) U.S. Cl. 604/154

(57) ABSTRACT

An injection device including a capacitor as an energy accumulator for providing energy for performing an injection.

### CROSS-REFERENCE TO RELATED APPLICATION(S)

This application is a continuation of International Patent Application No. PCT/CH02/00563, filed on Oct. 15, 2002, which claims priority to German Application No. 101 51 471.9, filed on Oct. 18, 2001, the contents of which are incorporated by reference in their entirety.

### BACKGROUND

The present invention relates to medical devices comprising an energy storage device, including those with a rechargeable energy storage device. Injection and infusion devices are types of medical devices to which the present invention relates. Such devices, including those referred to as injection pens or pens, have an associated energy storage capability, and/or are fitted with or carry energy storage devices to provide the energy required for an injection or for the delivery of a medicinal substance.

Injection devices may carry non-replaceable batteries, energy accumulators or energy storage devices to provide the energy required for an injection. A substance to be injected can then be introduced into a body, for example through the skin, using an injection device, usually via a needle, or also without a needle. For convenience, injection devices, particularly those known as pens, are often about as large as a writing implement and can simply be transported and operated by hand.

An inductive charging device for injection devices is known from DE 100 04 314 A by the Applicant.

Injection devices should in general be small and relatively lightweight. Batteries and/or other energy accumulators, however, demand a relatively large space in the injection device. Further miniaturizing accumulators leads to a deterioration of their charging properties; smaller batteries in particular have to be charged over a longer period of time than larger batteries—sometimes several hours—in order to be able to provide a reasonable amount of energy, for example as is known in mobile phones. A relatively large amount of energy is required to perform an injection. Thus, if a smaller accumulator were used, then it would have to be charged over a longer period of time if a number of injections are to be performed. Long charging processes and therefore preparation times before performing an injection leads, however, to acceptability problems for users of such an injection device.

Alternative methods of storing energy, such as for example by means of pressurized gas, are relatively elaborate with regard to the devices required and thus also cannot be suitably miniaturized.

### SUMMARY

It is an object of the present invention to provide an injection device which stores a sufficient amount of energy for performing at least one injection and which can be comparatively quickly charged.

In one embodiment, the present invention comprises an injection device comprising a capacitor for providing energy for performing an injection.

In accordance with one embodiment of the invention, a capacitor is provided in an injection device or an infusion device, either of which may or may not comprise a needle, the capacitor preferably exhibiting a relatively high capacity. Capacitors are relatively small and do not require an elaborate electronic system to be charged. As opposed to other energy accumulators, capacitors can be quickly charged within a relatively short time, e.g., a few seconds, using high currents, without compromising their service life as is the case with accumulators. Also as opposed to the aging of accumulators after a number of charging processes, also referred to as memory effect, capacitors have a constant quality even after many charging and discharging processes. In this way, injection devices with a longer service life can be created.

In some embodiments, the capacitor is advantageously designed such that it can store a sufficient amount of charge or energy to supply an electrical device for performing an injection, such as, for example, a magnet or an electric motor, with sufficient energy for at least one injection. Thus, an amount of energy can be stored which is sufficient to sufficiently accelerate an acceleration element required for injection. In some embodiments, a spring may be provided for this purpose, and may be charged by compressing the spring via an electric motor, powered as needed by using the discharge current from the capacitor. In general, an injection device in accordance with the present invention, i.e., one having an associated capacitor, should be able to store enough energy for it to be possible to dispense a substance to be injected using the injection device and to introduce it into a body, via a needle or without a needle.

In some embodiments, a gold capacitor is particularly preferably used as the energy storage device of the injection device. Gold capacitors can be manufactured to a high capacity, for example in the range of 1.0 to 10 F, such that by using such gold capacitors it is possible to store a sufficiently large amount of energy in the injection device within a relatively short time using a high charging current. Exemplary gold capacitors are available from Panasonic as duplex capacitors. Capacitance arrangements such as that disclosed in US publication 20030123215, the disclosure of which is incorporated herein by reference, might also be adapted for use in the present invention. Generally, any suitable capacitor or capacitance arrangement might be adapted for use in the present invention. Capacitors having a high capacity can also release high currents, such that energy-intensive processes, for example an injection, can be performed.

Other capacitors, such as for example electrolytic capacitors, double layer capacitors such as that disclosed in U.S. Pat. No. 5,986,876, which is incorporated herein by reference, or tantalum capacitors, such as that disclosed in U.S. Pat. No. 4,683,516, can also advantageously be adapted to store electrical energy in an injection device, providing a sufficiently high capacity can be provided using these capacitors. A number of capacitors of the same or of different designs can be connected in parallel as appropriate, in order to further increase the capacity provided.

In some embodiments, a charge indicator is preferably provided, by which the level of charge in the capacitor or capacitors can be indicated. When using a high-capacity capacitor as the one energy storage device in accordance with the invention, the amount of energy stored in the capacitor can be relatively precisely indicated, simply by measuring the voltage on the capacitor, wherein said measurement can substantially be taken without an appreciable measuring current. As opposed to charge indicators in accumulators, which indicate an almost complete level of charge over a wide operational range of an accumulator and indicate a relatively quick drop in the level of charge towards the end, it is possible in accordance with the invention to indicate precisely how much energy is still available, i.e., how many injections can still be performed.

In this respect, a circuit or other suitable processing and/or control device can advantageously be associated with an injection device in accordance with the present invention. The circuit or suitable processing and/or control device can determine, sense, and communicate or translate the number of injection processes for which there is still sufficient electrical energy stored in the capacitor. This determination can be made by measuring and/or assessing any parameter relative to injecting, such as for example the quantity of the material to be injected, the delivery force required, the frictional forces in the ampoule, the viscosity of the substance to be released, the length of the needle, the diameter of the needle, etc.

It would be advantageous if the number of injections which can still be performed is indicated, for example on an LCD or an LED display or by means of a number of adjacent LEDs, wherein, in some embodiments, the number of LEDs which light up once the display has been activated indicates the number of injection processes which can still be performed. The capacitor can also be used to supply current to the LCD or LED elements.

In some embodiments, a threshold value detector is advantageously provided which, when the voltage drops below a predetermined minimum voltage, outputs a signal that the amount of energy available has dropped below a predetermined value and is for example no longer sufficient for an injection. In some embodiments, the minimum voltage is advantageously set such that at least one injection process can definitely still be performed using a capacitor in which a voltage is above the minimum voltage.

In some embodiments, a voltage regulator, in particular a DC/DC converter such as one of those known in the electrical art, is preferably connected to the capacitor, such that a substantially constant DC voltage for operating the injection device, for example an electric motor associated with the injection device, can be obtained from the variable DC voltage on the capacitor. Buck converters and boost converters are known, using which a DC voltage can be obtained above or below the input voltage. A buck-boost converter or an inverting circuit regulator can equally be used.

In some embodiments, the at least one capacitor used is preferably connected such that it can be charged from an external energy source by inductive coupling. With respect to this, reference is made to application DE 100 04 314 A, the disclosure and teachings of which are incorporated herein by reference, with respect to designing a charging device, coupling an injection or infusion device to a charging device and designing the electronic system in the device to be charged. The principles of and mechanisms for inductive coupling may also be understood by reference to U.S. Pat. No. 6,648,914, the disclosure and teachings of which are incorporated herein by reference.

In some embodiments of the present invention, it may be particularly advantageous to connect the capacitor in series with a diode, preferably a power diode, and an induction coil, such that the capacitor is always charged with the desired polarity. The capacitor can, however, also be charged via contacts.

It should be appreciated that as well as for performing an injection, a high-capacity capacitor can also advantageously provide energy for storing data over a relatively long time or also for a signal output device, such as an optical or acoustic display.

### BRIEF DESCRIPTION OF THE DRAWINGS

FIG. 1 depicts, in representational form, one embodiment of the present invention.

### DETAILED DESCRIPTION

For details regarding one embodiment of the present invention, particularly one embodiment of an injection device, a charging device and an arrangement and method for inductive charging, reference is made to application DE 100 04 314 A, the disclosures and teachings of which, including the drawings, are incorporated herein by reference. In some preferred embodiments, an injection device in accordance with the present invention would carry a capacitor or capacitors rather than a battery.

Any suitable capacitor or capacitance arrangement, including those involving inductive charging, might be adapted for use in the present invention. Capacitance arrangements such as that disclosed in US publication 20030123215, the disclosure of which is incorporated herein by reference, may be adapted for use in the present invention. Other capacitors, such as for example electrolytic capacitors, double layer capacitors such as that disclosed in U.S. Pat. No. 5,986,876, which is incorporated herein by reference, or tantalum capacitors, such as that disclosed in U.S. Pat. No. 4,683,516, can also advantageously be adapted to store electrical energy in an injection device, providing a sufficiently high capacity can be provided using these capacitors. A number of capacitors of the same or of different designs can be connected in parallel as appropriate, in order to further increase the capacity provided.

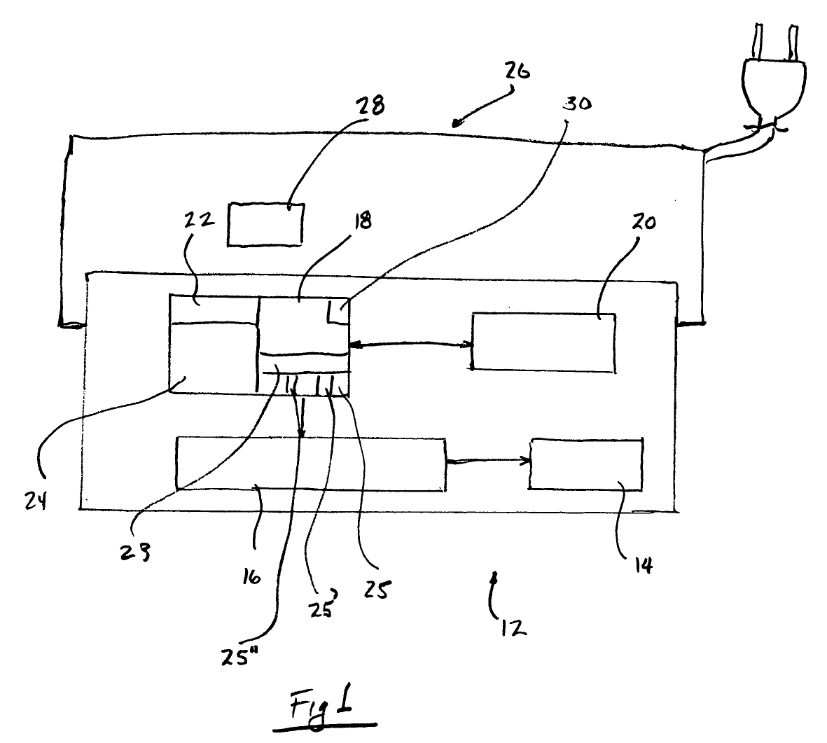

FIG. 1 depicts, in representational form, one embodiment of the present invention wherein an injection device 12 comprises a reservoir 14, a drive system 16, an electronic system 18, and an energy supply 20.

The reservoir 14 may take the form of a suitable ampoule, including one selected from interchangeable ampoules, disposable ampoules or a fluid or liquid containing cavity.

The drive system 16 may take the form of any suitable mechanical or electromechanical system, such as those known in the art of injection and infusion devices. Typical examples include systems wherein a piston is linked through a rod to driver, for example, a spring or a motor. Typically, in this type of drive system, the piston is moved to force a medicinal fluid, substance or liquid from the reservoir 14.

The electronic system 18 comprises suitable circuitry and components adapted for the purpose and selected as necessary to accomplish objectives and functions. In some embodiments, the system 18 includes inductive charging elements 22, sensing elements 23, suitable control/processing elements 24 (e.g., a microprocessor, memory, etc.) and display elements 25. The display elements may include a charge indicator 25′, for example a voltmeter, and a suitable display output such as one or more LED's 25″. Other components, such as a voltage regulator 30 (e.g., a DC/DC converter, etc.), are incorporated as required.

In some embodiments, the present invention encompasses a suitable charging device 26, including inductive charging elements 28.

In some embodiments, the energy supply 20 may comprise one or more capacitors of a suitable type. In some embodiments, the energy supply 20 is rechargeable, for example by inductive charging using the charging device 26.

In the foregoing description, embodiments of the present invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a capacitor as an energy storage device for performing at least one injection.

2. The injection device as set forth in claim 1, wherein the at least one capacitor is a gold capacitor.

3. The injection device as set forth in claim 1, wherein the at least one capacitor is a duplex capacitor.

4. The injection device as set forth in claim 1, further comprising a charge indicator operably coupled to the capacitor.

5. The injection device as set forth in claim 4, wherein the charge indicator is a voltmeter.

6. The injection device as set forth in claim 1, further comprising a processor for determining the number of injections which can be performed.

7. The injection device as set forth in claim 1, further comprising a threshold value detector operably coupled to the at least one capacitor to detect a predetermined minimum voltage which is the amount of energy at least one injection consumes.

8. The injection device as set forth in claim 1, further comprising a DC/DC converter operably coupled to the at least one capacitor.

9. The injection device as set forth in claim 1, wherein the at least one capacitor is adapted be charged inductively.

10. The injection device as set forth in claim 1, further comprising at least one of a memory and a signal output device, said at least one of the memory and the signal output device supplied with current from the at least one capacitor.
